(19) United States
(12) Patent Application Publication
(10) Pub. No.: US 2017/0273665 A1
(43) Pub. Date: Sep. 28, 2017

(54) Pose Recovery of an Ultrasound Transducer

(71) Applicant: Siemens Medical Solutions USA, Inc., Malvern, PA (US)

(72) Inventors: Ankur Kapoor, Plainsboro, NJ (US); Wen P. Liu, Plainsboro, NJ (US); Rui Liao, West Windsor Township, NJ (US); John Klepper, Seattle, WA (US)

(21) Appl. No.: 15/372,244

(22) Filed: Dec. 7, 2016

Related U.S. Application Data
(60) Provisional application No. 62/314,300, filed on Mar. 28, 2016.

(51) Int. Cl. A61B 8/00; A61B 90/00; A61B 8/08; A61B 8/12; A61B 6/12; A61B 6/00; A61B 1/313

(52) U.S. Cl. CPC A61B 8/4254; A61B 6/4417; A61B 90/39; A61B 1/3132; A61B 8/4483; A61B 8/12; A61B 8/4263; A61B 6/12; A61B 8/461; A61B 8/4444; A61B 8/5215; A61B 2090/3966

(57) ABSTRACT

Pose of an ultrasound transducer is recovered. In one approach, inertial measurement units are positioned on the ultrasound transducer. The measurements from the inertial measurement units are used with pose or measurements from another position sensor (e.g., x-ray, electromagnetic, or optical) to improve accuracy and/or provide pose information at a greater rate. In another approach, a curve, line, or other connected shape of light emitting diodes are incorporated into the transducer. Optical tracking, with a filter specific to the light emitting diodes, using the connected shape pattern is used to determine the pose.

### RELATED APPLICATIONS

The present patent document claims the benefit of the filing date under 35 U.S.C. §119(e) of Provisional U.S. Patent Application Ser. No. 62/314,300, filed Mar. 28, 2016, which is hereby incorporated by reference.

### BACKGROUND

The present embodiments relate to ultrasound imaging. In particular, a pose of an array used in ultrasound imaging is determined.

Tracking systems are used in diagnostic image acquisitions and in image-guided interventions. Tracking systems provide an estimate of the position and orientation (pose) of the array in the clinical environment.

Electromagnetic tracking solutions generate magnetic fields to triangulate distances between transmitters and coil sensors. For the case of the ultrasound catheters that navigate within the body cavity without line of sight access to reflective markers, electromagnetic tracking is most predominant. Optical infrared tracking solutions require strict line-of-sight access to reflective markers, which may be occluded. Typically, the accuracy of electromagnetic solutions is less than optical solutions as local field distortions are caused by ferromagnetic objects. These electromagnetic and optical tracking solutions cost thousands of dollars.

For catheters, another approach is to embed radio-opaque markers and detect the distribution of the markers with fluoroscopy imaging. Such an approach is practical as these ultrasound catheter devices are often jointly used with fluoroscopic guidance. However, it is well known that the accuracy of pose recovery using a pattern of markers (e.g., 6 or more radio-opaque beads) is dependent on the magnitude of the smallest dimension of the pattern. For an ultrasound catheter, the smallest dimension may be of the order of 3-4 mm due to the physical bounds of the catheter. As a result, a theoretical limit is placed on the achievable accuracy of pose recovery. Another drawback of this approach is the amount of radiation involved since device tracking relies on X-ray imaging with ionized radiation.

### SUMMARY

By way of introduction, the preferred embodiments described below include methods, systems, instructions, and computer readable media for pose recovery of an ultrasound transducer. In one approach, inertial measurement units are positioned on the ultrasound transducer. The measurements from the inertial measurement units are used with pose or measurements from another position sensor (e.g., x-ray, electromagnetic, or optical) to improve accuracy and/or provide pose information at a greater rate. In another approach, a curve, line, or other connected shape of light emitting diodes are incorporated into the transducer. Optical tracking, with a filter specific to the light emitting diodes, using the connected shape pattern is used to determine the pose.

In a first aspect, a system is provided for pose recovery of an ultrasound transducer where an inertial measurement unit connects with the ultrasound transducer. A position sensor is positioned to sense the ultrasound transducer. A processor is configured to determine the pose of the ultrasound transducer based on outputs from the position sensor and the inertial measurement unit.

In a second aspect, a system is provided for pose recovery of an ultrasound transducer where light emitting diodes are positioned on the handheld housing of the ultrasound transducer. The light emitting diodes form a geometric pattern of adjacent, visually connected light sources. A camera is positioned to capture an image of the light emitting diodes. A filter is configured to reduce signal in the image at frequencies different than emission frequencies of the light emitting diodes. A processor is configured to determine the pose based on minimization of difference between the image output by the filter and two-dimensional projection from a model of the geometric pattern.

In a third aspect, a system is provided for pose recovery using catheter with an array of acoustic transducer elements, an inertial sensor, and a radio-opaque marker. An x-ray imager is configured to image the catheter while in a patient. A processor is configured to determine a pose of the array with the image from the x-ray imager and an output of the inertial sensor.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for pose recovery of an ultrasound transducer;

FIG. 2 illustrates two example arrangements of radio-opaque markers and inertial measurement units on ultrasound catheters;

FIG. 3 illustrates six example patterns of connected shapes formed by light emitting diodes;

FIG. 4 is a table of example accuracies in pose provided by optical detection of the shapes of FIG. 3;

FIG. 5 shows an example handheld transducer with embedded light emitting diodes and corresponding optical capture of the pattern;

FIG. 6 shows an example laparoscope incorporating embedded light emitting diodes and an inertial measurement unit;

FIG. 7 shows another example laparoscope incorporating an inertial measurement unit;

FIG. 8 shows an example position sensor based on wireless radio-frequency transmissions;

FIG. 9 shows an example processor implementation of pose recovery using a position sensor and an inertial measurement unit;

FIG. 10 is a table of example accuracies using point fiducials, pattern fiducials, and a combination of pattern fiducials and inertial measurements;

FIG. 11 shows an example processor implementation of pose recovery using multiple position sensors and an inertial measurement unit;

FIG. 12 shows an example processor implementation of pose recovery using optical detection of light emitting diodes;

FIG. 13 is a flow chart diagram of one embodiment of a method for recovering pose using a combination of x-ray imaging and inertial measurement; and

FIG. 14 is a flow chart diagram of one embodiment of a method for recovering pose using light emitting diodes.

### DETAILED DESCRIPTION OF THE DRAWINGS AND PRESENTLY PREFERRED EMBODIMENTS

In one approach to pose recovery, an inertial measurement unit (IMU) is used with other position sensors. For example, the IMU includes multiple micro-electro-mechanical systems (MEMS) and is used in conjunction with X-ray imaging of a specific radio-opaque pattern embedded on an ultrasound catheter to provide an accurate and robust estimate of the pose of the ultrasound catheter. While IMUs are increasingly accurate in providing the orientation, the position recovery using just the integration of linear accelerations obtained from these MEMS sensors may not be sufficient for the purposes of guidance in clinical environments. The multi-sensor fusion overcomes this limitation by incorporating additional input from pose recovered by X-ray imaging of a specific radio-opaque pattern or other position sensing. Using one or more IMUs is relatively compact, cost-effective (orders of magnitude less), highly accurate, and robust. With the advent of smart consumer products (i.e. smart phones, watches, etc.), high performance sensors and devices, such as inertial measurement units (IMU), have become inexpensive off-the-shelf sensors.

X-ray imaging is obtained only intermittently. Thus, there may be temporal gaps during the clinical procedure when the pose recovery from X-ray imaging may not be available. Further, during this period, the device may have moved significantly, resulting in loss of tracking in subsequent X-ray images. Additional sources for positional information may be incorporated to fill the temporal gaps. One such source is decorrelation of ultrasound data itself. Another source is a wireless based indoor positioning system which provides positional information based on triangulation of narrow band RF signals. Alternatively or additionally, the IMU provides pose information in the temporal gaps. Bi-directional information sharing between multiple pose recovery approaches may result in an overall accuracy that cannot be delivered by individual approaches alone.

In another approach that may be used with or without the fusion of multiple position sensors, a commercial camera observes an illuminated compact light emitting diode (LED) pattern. A monocular vision-based pose recovery system tracks the three-dimensional (3D) position and orientation of medical devices (i.e. ultrasound probes, pointers, needles, etc.). An illuminated compact LED pattern, affixed to an ultrasound transducer, is observed by a single camera equipped with a color band-pass filter. The filter eliminates or reduces signal at frequencies outside of a narrow band, centered at the LED color, so is more robust to image noise and illumination change when compared to cameras tracking in the entire visible spectrum. Duplicate camera and target configurations may be added to extend the system from monocular into a stereo. Multi-camera setups with redundant views of the target pattern may improve performance and robustness to clutter. Multiple targets may be tracked concurrently by using unique patterns and different color frequencies and filters.

A camera and LED pattern approach may be a robust, inexpensive alternative that achieves required clinical accuracy while addressing the disadvantages of current clinical systems. Compared to traditional electromagnetic and optical systems, the commercial camera and LED solution is relatively compact, costs orders of magnitude less, and may be configured to overcome occlusion and different clinical environments.

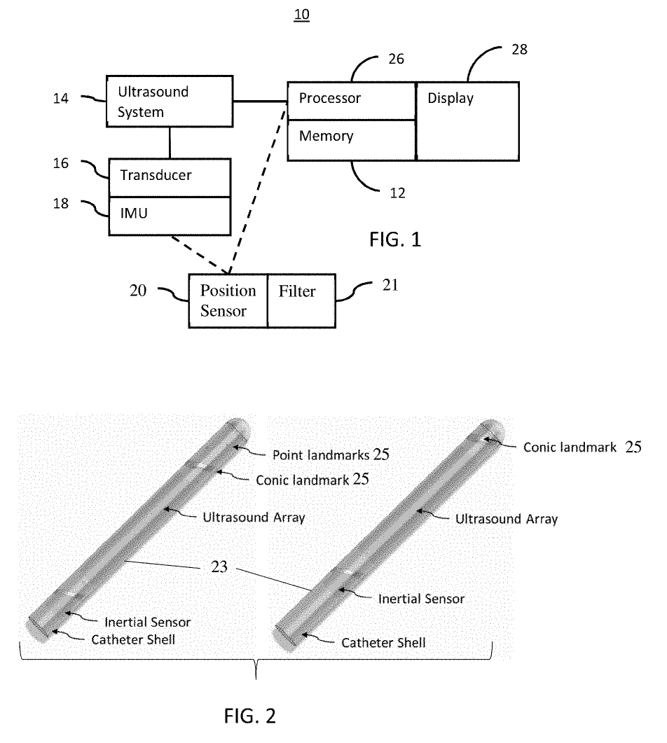

FIG. 1 shows a system 10 for pose recovery of an ultrasound transducer 16. The system 10 includes a memory 12, an ultrasound system 14, the transducer 16, an IMU 18, a position sensor 20, a processor 26, and a display 28. Additional, different, or fewer components may be provided. For example, a network or network connection is provided, such as for networking with a medical imaging network or data archival system. As another example, a user interface is provided. In yet another embodiment, a system for scanning with a different modality (e.g., magnetic resonance or computed tomography system) is provided. The ultrasound system 14 may not be provided in some embodiments, such as where a needle, non-ultrasound catheter, or another device is tracked. In yet another embodiment, the IMU 18 is not provided.

The processor 26 and display 28 are part of a medical imaging system, such as the ultrasound system 14, other image modality system, or another system. Alternatively, the processor 26 and display 28 are part of an archival and/or image processing system, such as associated with a medical records database workstation or server. In other embodiments, the processor 26 and display 28 are a personal computer, such as desktop or laptop, a workstation, a server, a network, or combinations thereof.

The ultrasound system 14 is any now known or later developed ultrasound imaging system. For example, the ultrasound system 14 is a cart-based, portable, or another medical diagnostic ultrasound scanner. In another example, the ultrasound system 14 is a therapeutic ultrasound system. The ultrasound system 14 includes one or more transducers 16 for converting between acoustic and electrical energies. The transducer 16 releasably connects to a port on the ultrasound system 14.

The ultrasound system 14 is configured by software, hardware, and/or firmware to acquire one or more frames of ultrasound data representing the patient. Transmit and receive beamformers relatively delay and apodize signals for different elements of an array in the transducer 16. Acoustic energy is used to scan a plane and/or volume. For example, a volume is scanned by sequentially scanning a plurality of adjacent planes. Any format or scan technique may be used. The scanned volume may intersect or include all the patient volume. B-mode, Doppler, or other detectors detect information from the beamformed signals. A scan converter, memory, three-dimensional imaging processor, and/or other components may be provided. The ultrasound data is output in a polar coordinate or scan converted Cartesian coordinate format.

The transducer 16 includes a one-, two-, or multi-dimensional array of piezoelectric or capacitive membrane elements. In one embodiment, the array is a curved linear or phased array. The transducer 16 is used to scan the patient with ultrasound.

The transducer 16 has any of various form factors or is one of various types of ultrasound probes. In one embodiment, the transducer 16 is an intracardiac echocardiography catheter. A long flexible catheter incorporates the array of acoustic transducer elements on a distal end. The catheter also includes other components for pose recovery. In the examples of FIG. 2, the catheter 23 includes an IMU, the array, and a plurality of radio-opaque markers or fiducials included as x-ray detectable landmarks.

The radio-opaque markers (25) are formed in a pattern on the catheter. The radio-opaque pattern is inked on to the ultrasound array assembly either via a pad printing, screen printing, inkjet, or thermal transfer. In one embodiment, this pattern is formed via a direct transfer of the radio-opaque material on the ultrasound array sub-assembly using a flow based micro-dispensing technique in which radio-opaque material is extruded through a syringe or similar tip. This technique directly produces three-dimensional geometries used for the pattern (e.g., three dimensional conics). Embedded structure or other formation may be used.

The radio-opaque pattern is formed of point landmarks (e.g., spherical beads) that are opaque to X-ray energies and hence appear dark in fluoroscopic images. Accurate pose recovery using the point landmarks may be limited by the smallest dimension of the pattern. For an ultrasound catheter, the diameter of the catheter is in the order of 3-4 mm. Geometric markers (25) (e.g., conics, ellipses, or cylinders) may be used. The portion of the ultrasound array at the distal tip is kept free of radio-opaque materials to provide an acoustic window for ultrasound imaging. In the examples of FIG. 2, the radio-opaque patterns are in two parts at either end of the ultrasound array. Two conics and two beads are at either end. Any combination of conics, points and lines may be deployed on either end of the pattern. The minimal pattern for successful pose recovery with the aid of an IMU is one bead at each end or one conic at each end, while more complex patterns provide more flexibility and robustness with respect to partial occlusion and image noises for pose recovery. The location of each of these parts with respect to each other as well as to the ultrasound imaging array is known either by calibration or by mechanical fabrication constraints.

In another embodiment, the ultrasound transducer 16 has a handheld housing, such as for a probe used for manual scanning from an exterior of a patient. The housing is shaped to be comfortably held by the sonographer. An acoustic window is provided adjacent to the array, so that the sonographer places the acoustic window against the patient to scan. The probe may be wireless. Alternatively, a cable connects the probe to the ultrasound system 14.

The handheld housing may include LEDs. The LEDs are embedded within the housing, such as being in a groove or holes or being formed in the housing. The LEDs are in the external casing or the external cable or strain relief assembly. The LEDs being embedded provide for a smooth or flat outer surface of the housing without bumps or protrusions. Alternatively, the LEDs are in a part that connects to the outside of the handheld housing. The grip or portion held by the user is at a location different than the LEDs to minimize occlusion.

Since the surface of the housing varies in 3D, the pattern of LEDs has a 3D distribution. Alternatively, the LEDs may be embedded in a planar portion of the housing. The LED pattern extends around 90-360 degrees about a center axis of an upper portion of the housing in one embodiment.

The LEDs are each a point source of light. Alternatively, a light guide, lens, diffuser, or other structure is used to cause the LEDs to emit light from the housing in a non-point shape, such as a line, curve, or area (i.e., 2D) shape of interconnected light. Either by placement adjacent each other (e.g., touching or within one width of an LED) or by light diffusion structure (e.g., a semi-transparent cover), the LEDs form a pattern as a line, curve, or other area shape distributed in one, two, or three dimensions on the housing. A geometric pattern with no or only some point sources of light included in the pattern is formed by the LEDs. The geometric pattern of adjacent LEDs results in a visual appearance of one or more connected light sources.

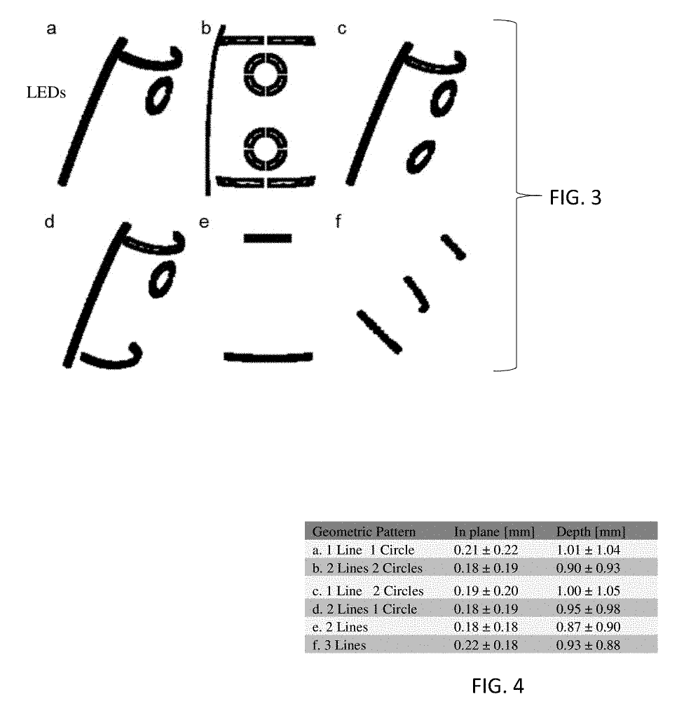

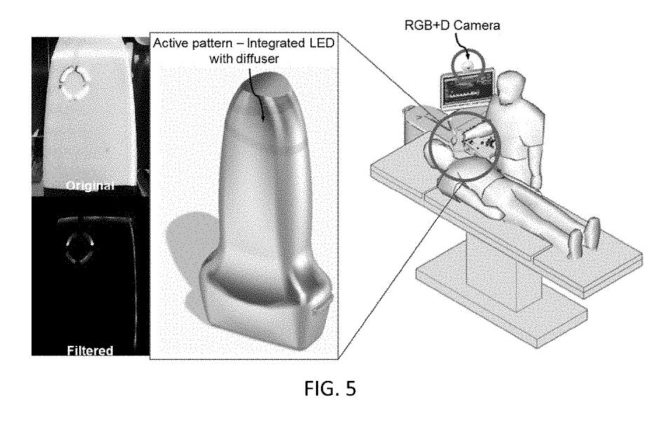

The design of the illuminated pattern is flexible and may be modified to accommodate the ergonomics of the transducer 16. FIG. 3 shows a variety of simple geometric patterns (i.e., lines, circles, ellipses, etc.) These patterns and their error analysis from experiments simulating 1000 randomized pose recoveries are summarized in FIG. 4. Using a computer-aided design (CAD) model of the probe, an inlaid translucent pattern is created using a 3D printer and back illuminated with green LEDs that can be integrated into the upper half of a linear abdominal ultrasound probe (middle of FIG. 5). A RGB depth camera is used to capture the transducer with the LED pattern. In the example of FIG. 5, the camera is positioned for capturing the transducer 16 for transcervical image acquisition readied for a head and neck procedure.

The experiments use a depth range of 500 mm to 700 mm; pitch ±75°; roll ±20°; yaw ±20°; and Gaussian blur noise. In the left-most column of FIG. 5, the top photograph shows an image captured by a 1600×1200 pixel camera of an LED pattern before (top) and after a 520 nm Center Wavelength band-pass filter 21 (10 nm full width at half maximum pass region)(bottom).

The LED pattern is a 3D illuminated object, with a unique pattern visible in 360° around the transducer 16, unlike most rigid body markers that begin to self-occlude at ±90°. Compared to optical tracking relying on point source signals, where the interference of a single marker point results in non-recoverable pose of the target object, a rigid transform of the illuminated pattern may be recovered using only a partial view.

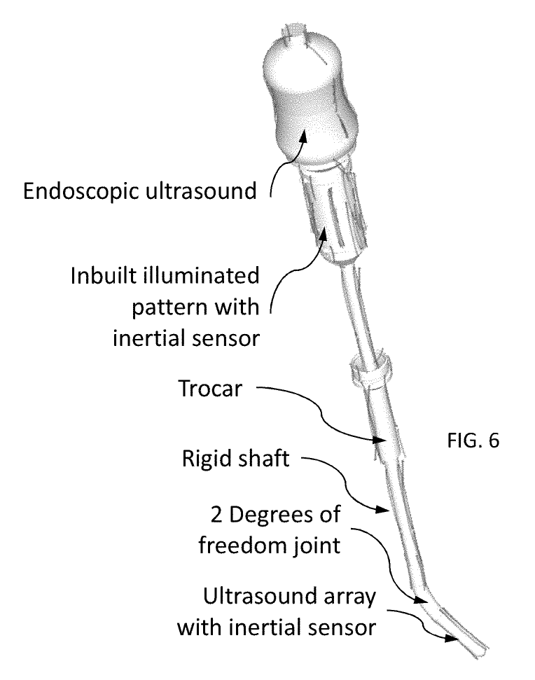

FIG. 6 shows another embodiment of the transducer 16. The transducer 16 is a laparoscope. Part of the laparoscope is inserted within the insufflated body cavity, not openly visible. Trans-esophageal, intraoperative, or other ultrasound probes may be used.

The laparoscope includes an LED pattern on a handheld portion. The handheld portion is rigidly connected to a rigid shaft. The tip or distal end includes an ultrasound array. The distal end may be rotated relative to the rigid shaft. An IMU is in or on the distal end. The distal end and part of the rigid shaft are insertable into a patient for scanning the patient from within the patient. The LEDs and handle remain outside of the patient.

The pose (e.g., location and orientation) of the array is determined by a combination of optical and IMU measurements. The LED pattern has a known position and orientation relative to the laparoscope. The IMU is a micro-electro-mechanical sensor (MEMS) chip located in the flexible portion that would be in contact with the organ of interest during a procedure. The IMU outputs angular measurements. One or two cameras are mounted outside of the surgical field to provide the information for external relative positioning of the laparoscope, using fiducial markings (e.g., LED pattern) placed on the shaft and handle. One or more IMU chips placed at either end of the array provide angular change information from a (0,0) calibration point. The angular change after the array is first inserted in the body cavity is measured. The gyro output (i.e., angle relative to gravity) and magnetic output (i.e., angle relative to earth's mag field) may be used to compute the location of the array in space relative to the LED positioned handle and rigid shaft. In other embodiments, other position sensors are used on the handle outside the body and/or by the array in the body.

Referring to FIG. 1, the IMU 18 connects with the ultrasound transducer 16. The connection is fixed, such as being mounted within the housing of the transducer 16. The connection may be releasable, such as being clipped onto the transducer 16. In the embodiments of FIGS. 2 and 6, the IMU 18 is within the catheter 23 and under the housing. The IMU 18 is adjacent to the array of transducer elements, such as within 1 cm of an edge of the array. More than one IMU 18 may be provided. Locations further from the array may be used, such as to determine relative rotation or location for other parts of the transduce 16. IMUs 18 may be provided for different parts of the transducer 16, such as parts moveable relative to each other. In the embodiment of FIG. 5, an IMU 18 may be added on or in the transducer 16.

The IMU 18 is a MEMS device, but may be formed from separate components. Any accelerometer, gyroscope, and/or magnetometer may be used. In one embodiment, the IMU 18 includes accelerometers for measuring linear motion along three orthogonal axes and gyroscopes for measuring rotational motion about three orthogonal axes. A magnetometer for measuring orientation relative to the magnetic poles of the earth may be provided. In some embodiments, only accelerometers are used. In such embodiments, a combination of three or more 3-axis acceleration sensors provide nine or more measurements for redundancy. Any suitable integration scheme may be used to obtain the pose estimation from acceleration and/or angular velocity measurements.

Temporal filtering using either an FIR or IIR-type filter 21 may be employed to improve signal-to-noise ratio of the pose estimates or measurements from the different components of the IMU 18. The measurements from the different sensors may be fused or combined. This process results in an estimate of the pose at one time with respect to a pose at another time. The sensor fusion provides a robust estimate of the orientation of the device with respect to an arbitrary coordinate frame from the gyroscopes and magnetometer. In addition, a robust estimate of the linear acceleration of the device after removal of acceleration from gravity may be provided. A rate-of-turn may be estimated.

In the embodiment of FIG. 2, the pose (position and/or orientation) of the array of the ultrasound catheter 23 is accurately determined in a clinical environment using, in part, IMU 18. Such an environment may include an X-ray system used to guide the ultrasound catheter 23 inside the body cavity. Due to presence of large metal objects and sources of magnetic disturbances (e.g., X-ray detector) electromagnetic and optical methods of tracking and pose estimation may not be sufficiently accurate to provide sub-millimeter and sub-degree position and orientation estimates. Likewise, the accuracy of pose recovered by use of X-ray radio-opaque patterns is limited due to the small size of the pattern embedded into an ultrasound catheter 23. The measurements and/or estimates from the IMU 18 fused with pose from other approaches (e.g., X-ray images, ultrasound data and/or wireless narrowband signals) may increase accuracy. Each information source has its own advantages and disadvantages in terms accuracy, speed, and/or robustness in providing pose estimation in a given parameter space (e.g., translation, rotation, and/or depth). By fusing all the information from different types of sources, pose estimation of ultrasound catheters 23 may be performed reliably and efficiently with a flexible and cost-effective configuration. Multiple sources of pose information may be used to assist in other embodiments, such as the laparoscope of FIG. 6. The LED and camera source of FIG. 5 may be used.

The position sensor 20 of FIG. 1 is an additional source or different source of pose information than the IMU 18. The position sensor 20 is positioned to sense the ultrasound transducer 16. Depending on the embodiment, the positioning may provide for line of sight with minimal occlusion (e.g., positioned on a ceiling directed downward or worn on a head of the sonography). The positioning may be on the table on which the patient rests, on a stand, on an imaging system, or at another location.

In one embodiment, the position sensor 20 is an x-ray imager. An x-ray source is positioned on one side of the patient and an x-ray detector is positioned on another or opposite side of the patient. The generated x-rays pass through the patient and ultrasound transducer 16, but are more strongly attenuated by the radio-opaque markers (25) on the ultrasound transducer 16. For example, by x-ray imaging the catheter 23 while in a patient, the markers (25) in the x-ray image may show a pose of the array.

In another embodiment, the position sensor 20 is a camera. Any camera may be used, such as a digital frame camera (e.g., charge coupled device), a line camera, or a depth camera (RGBD). The camera captures an image of the transducer 16, from which the pose may be determined. More than one camera may be used, such as a stereo camera arrangement.

For use with LEDs, the position sensor 20 is a camera with a filter 21 configured to isolate information from a range of frequencies of the LEDs. The filter 21 is implemented by a circuit or processor, so may be in a component housed with or separate from the camera. The camera captures an image of the LEDs, and the filter 21 reduces signal in the image at frequencies different than emission frequencies of the LEDs (compare the captured image on the upper right of FIG. 5 with the filtered image on the lower right of FIG. 5).

In another embodiment, the position sensor 20 is the ultrasound scanner or system 14. The correlation between any two ultrasound acquisitions is a function of the distance and orientation between the frames. For a volumetric ultrasound transducer, its relative position between two consecutive acquisitions may be established via 3D-to-3D volumetric image registration between the two acquired echo volumes. Where the overlapping region of the volumes is sufficiently large, the registration indicates an accurate alignment.

For a 1D ultrasound transducer, given a pair of corresponding patches in the two 2D acquisitions, which may contain coherent and non-coherent signals, a calibration curve relating the 1-degree of freedom (df) out-of-plane distance between the patches to the correlation coefficient between the patches may be established. For example, a calibrated model using Rician-Inverse Gaussian (RiIG) stochastic process of the speckle formation may be used to obtain this curve. A voting-based approach (e.g., RANSAC) may be used to determine the 6-df rigid motion between the two frames using a number of such 1-df measurements.

In one embodiment, a selection of a number of small patches is used. 1-df motion of each of these patches between consecutive frames is computed. The estimated motion of the ROI is the motion that is the consensus of most the patches. To suit acquisition images that have changing image intensities in the target ROI, sample patches from both within and outside the ROI are used. Patches sampled from outside the ROI serve as reference, and indicate when patches inside the region need updating. Patches whose motion is consistent with the majority of data are retained, whereas others are pruned and replaced by an equal number of patches sampled from the respective regions, and the process is continuously repeated. This process results in an estimate of the pose of a frame with respect to another. Thus, the pose of the i^{th} frame may be computed by chaining the relative poses between the 0^{th} and (i−1)^{th} frames. Due to the cumulative nature of this computation, the error in pose also increases with each subsequent frame. Further, the calibration curve only provides an absolute value of distance and does not include the direction of motion between frames.

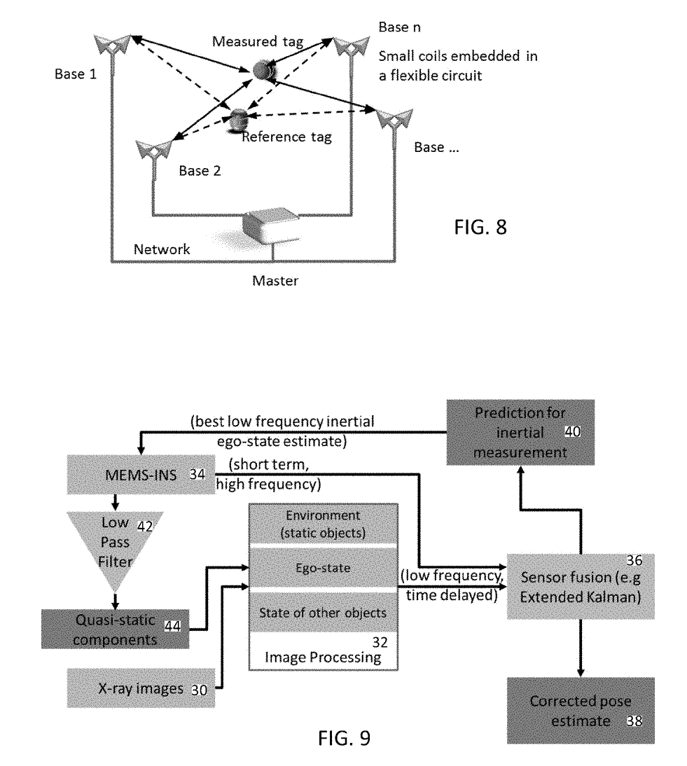

In another embodiment, the position sensor 20 is a position sensor or receiver of an indoor wireless position sensor (e.g., indoor “GPS”). FIG. 8 shows one example. Each base transmits coded radio frequency signals, such as at ultra-wide-band frequencies. The tags are antennas and receivers that receive the signals and process the signals to determine distances from the bases. The position is provided by triangulation. Finding the position at multiple locations may improve accuracy. The accuracy of such technologies is currently limited to fraction of meters (approx. 0.3 m). If measurements are made with respect to another body in the same region of interest, the accuracy of measurement is in the order of 2-5 mm. FIG. 8 shows a reference tag for this purpose. The reference tag is kept stationary and acts as a reference at a known position. The other tag is attached to the ultrasound transducer 16. In the case of wireless transducer, the transducer wireless communication channel itself may be utilized as the receiver for distance computation.

The processor 26 is a general processor, central processing unit, control processor, graphics processor, digital signal processor, three-dimensional rendering processor, image processor, application specific integrated circuit, field programmable gate array, digital circuit, analog circuit, combinations thereof, or other now known or later developed device for recovering pose of an ultrasound transducer. The processor 26 is a single device or multiple devices operating in serial, parallel, or separately. The processor 26 may be a main processor of a computer, such as a laptop or desktop computer, or may be a processor for handling tasks in a larger system, such as the ultrasound system 14 or position sensor 20.

The processor 26 is configured by software, firmware, and/or hardware. The processor 26 is configured to determine the pose of the ultrasound transducer 16. One or more of the IMU 18 and/or position sensor 20 is used to determine pose.

The pose is determined once or in an on-going manner, such as tracking the absolute pose and/or changes in the location and/or orientation. The same or different set of IMU 18 and/or position sensor 20 is used for determining an initial position. The processor 26 is configured to initialize the determination of the pose. The initialization provides an absolute pose or provides an indication or estimate of the pose from which the absolute pose is then determined.

Different initialization may be used for different embodiments. For an example in determining the pose of the array in a catheter 23 using the IMU 18 and an x-ray imager as the position sensor 20, the current pose may be initialized using a prior pose estimation or by applying a motion model to the prior pose. The motion model is either from a learned filter (e.g., expectation maximization, such as a Kalman filter) or derived from linear acceleration values obtained from the IMU sensor 18. Another approach uses a multi-detector arrangement so that x-ray projections from different angles are obtained. The initial pose is determined using multiple images, resulting in resolving depth ambiguity of x-ray imaging by back projection. The depth estimate may have some uncertainly due to factors such as errors in calibration of detectors and physiological motion. Other initialization may be used.

The IMU 18 and x-ray-based system is calibrated. The IMU 18 provides measurements with respect to a fixed but arbitrary coordinate system, whereas the pose recovered from X-ray imaging is with respect to the x-ray system's own fixed coordinate system. The x-ray coordinate system is typically calibrated about the isocenter of C-arm rotation. The relative transformation between these two reference coordinate frames is determined at least once for each C-arm system as a calibration. A calibration may be performed by attaching an IMU 18 to a calibration phantom with a radio-opaque pattern in a known mechanically constrained location. The radio-opaque pattern is designed sufficiently large to cover the field of view under typical operating conditions and is imaged in multiple poses to increase the accuracy of calibration. Since in a typical clinical scenario, the pose of the ultrasound image is determined with respect to X-ray instead of the pose of the catheter device, another calibration between the reference coordinates of the IMU 18, X-ray system and the ultrasound image (e.g., array with known position to the ultrasound image) is performed. This calibration may be accomplished by using a multi-modality calibration phantom. Other calibration may be used.

In the LED-based pose recovery system of FIG. 5 or the laparoscope embodiment of FIG. 6, the processor 26 initializes the pose. The current pose may be initialized using the prior pose estimation or by applying a motion model derived from linear acceleration values from IMU 18 to the prior pose. In another approach, the camera is a depth or stereo camera, so captures a depth map. A 3D point cloud of the transducer 16 and/or a hand holding the transducer 16 is provided as a depth map (i.e., distances from the camera to the visible surface at various locations). The depth map may be used to determine the pose of the transducer 16. Other initialization methods may be used.

The LED-based system is calibrated. Any camera calibration may be applied to obtain the optical parameters used in pose recovery to generate a 2D projection of the 3D LED model. Optionally, if multiple cameras are configured, calibration may require either a one-time process for a static setup or online self-calibration by correspondence. For establishing correspondence, methods in point-to-point and feature-based approaches may be used. Ultrasound-to-video calibration registers the ultrasound and video coordinate systems. A multi-modality phantom, with features visible in both ultrasound and in video, may be used to establish direct point-to-point correspondence. Other calibration methods may be used.

The processor 26 is configured to determine the pose of the ultrasound transducer 16 based on outputs from the position sensor 20 and/or the inertial measurement unit 18. The pose is determined as an initial pose or subsequent pose. The pose is the position in one or more degrees of freedom (e.g., 3) and/or the orientation in one or more degrees of freedom (e.g., 3). The scale is determined based on calibration, but may alternatively be determined by the processor 26 from IMU 18 and/or position sensor 20 outputs.

In one embodiment, the pose is determined from image processing of an image from the x-ray imager as the position sensor 20. The pose is determined as a minimization of difference between the image output by the x-ray imager and two-dimensional projection from a model of the geometric pattern of the radio-opaque markers (25). The x-ray image may be spatially filtered and/or filtered by thresholding or other process to highlight the markers (25) and/or reduce signal from structures other than the markers (25). The model of the markers (25) is rendered or projected to emulate x-ray images from different view directions. By finding the projection of the model that best matches the x-ray image of the actual catheter 23, the pose is determined from the view angle and position of the matching model projection. The model may be a computer-generated physics model or may be x-ray images of the catheter 23 in a phantom or not in the patient but from different angles.

Any pose recovery may be used. For example, template patterns from different poses are compared to the measurements. As another example, a machine-learnt classifier or Kalman filter (or other type of expectation maximization) recovers the pose based on the input measurements.

In one embodiment, the recovery of the pose of the marker (25) pattern is formulated as an optimization problem with the following non-convex formulation:

min_{xεSE}_{³}ƒ(x) (1)

where ƒ(x) is an objective function, and x are transformation parameters that are a parameterization of special Euclidean group SE³. Any representation of the N (e.g., N=6) degrees of freedom of SE³ space may be used to parameterize the transformation T(x)εSE³. The parameters x may be decomposed into rotational parameters, xᵣ and translational parameters, xₜε [symbol] ³. A unit quaternion xᵣε [symbol] ⁴ or Euler angle xᵣε [symbol] ³ is used to represent xᵣ, but other representations may be used.

In one embodiment, the function ƒ(x) is described by:

ƒ(x)=∥I−M(T(x))∥ₚⁿ (2)

where model M is transformed by rigid transformation T(x)εSE³ parameterized by x. The filtered image output by the x-ray imager is given by I. A p-norm is used, where p can be 1, 2 or infinity. For values of p=2 and n=2, the function is reduced to the sum of differences square (SSD) metric, as represented by:

ƒ(x)=∥I−P(M(T(x))∥ₙ^{p} (3)

An operator P models the accumulation of X-ray attenuation of the pattern and is applied to provide a more realistic projection of the model M for measuring the matching. Other measures of similarity of the projection of the model at different angles and location (e.g., different rigid transformations) may be used, such as the sum of absolute differences (e.g., n=1) or a correlation,

In another embodiment, a distance weighted match is used, as represented by:

ƒ(x)=∥D(Iₛ)−D(T(x)))∥ₚⁿ (4)

where Iₛ is a skeletonization of the filtered x-ray image, I. A distance map is computed by function D(•). The distance map function transform each point in the input image into a floating-point number dependent on the distance between the image point and the nearest point on the skeleton of the filtered image. The distance mapping is also applied to the transformed or projected model. Euclidean distance norms are used, but fractional powers of Euclidean distance norms or other distance measures may be used. Other approaches to matching may be used.

In one embodiment, the objective function for the LED and camera position sensor 20 accounts for lighting effects. Rather than using an attenuation model, a model, G, of light diffusion is applied, as represented by equation (3) with P replaced by G:

ƒ(x)=∥I−G(xᵣ)*M(T(x))∥ₙ^{p} (5)

The light diffusion of the pattern is applied to provide a more realistic projection of the model M. The operator G may be dependent on the rotation parameters of the pattern. The operator G may be implemented as a convolution by a Gaussian or Laplacian kernel, but other diffusion modeling may be used. The skeletonization approach may additionally or alternatively be applied.

Where multiple detectors of position (e.g., multiple x-ray images from different directions) are used, equation (1) may be modified to equation (5):

min_{xεSE}³Σᵢ₌₁^{N}ƒ(x); s·t·hᵢⱼ(x)=0 ∇ i,j=1 . . . N; i≠j (6)

In this multiple detector case, the objective function, f(x), is a linear combination of the objectives for each of the detectors. Optionally, Epiploar constraints between x-ray detector pairs i, j may be incorporated as hᵢⱼ(x). Using of multiple detectors offers better accuracy and robustness to occlusion.

In other embodiments, the multiple detectors are of different types, such as the x-ray imager and an IMU 18. The pose provided by the objective function for each detector or type is combined. Other combinations of IMU 18, x-ray imager, ultrasound system 14, indoor wireless, optical (e.g., LED-based or other), or electromagnetic may be used.

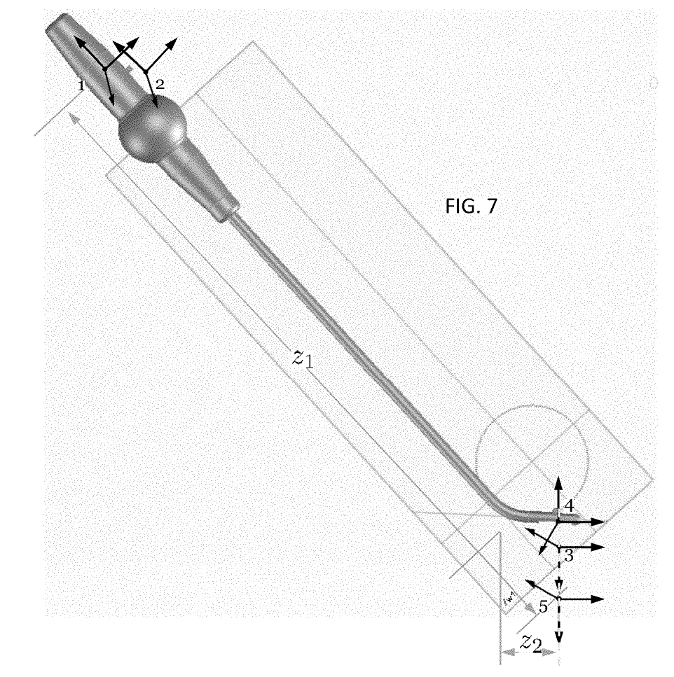

FIG. 7 shows one embodiment to solve for the pose of the laparoscope of FIG. 6. The processor 26 is configured to determine pose of the handle using an optical pattern (labeled as coordinate system 1) and IMU measurements form an IMU on the handle (labeled as coordinate system 2), pose of a center line of the ultrasound image (labeled as coordinate system 3), pose of the laparoscope on the distal end using an IMU (labeled as coordinate system 4), and the center of the ultrasound image (labeled as coordinate system 5). The pose of the array (translation and angulation), ¹T₃, depends on the angular manipulation of the array, and is given by:

¹T³=[¹R₃,¹p₃]=T_{z}_{₂}R(α,β)T_{z}_{₁} (7)

where p is a position, R is rotation, and the superscripts represent the coordinate system in which the measurements are made and the subscripts represent the coordinate system whose measurements are being estimated, ¹T₃ is provided by tracking of the optical pattern or based on the optical pattern and IMU measurements (e.g., sensor outputs or fused information from the IMU). ¹R₃, ¹p₃ is given by:

¹R₃=R(α,β)&¹p₃=z₁R(α,β)z₂ (8)

z₁ and z₂ are values set during assembly or measured as part of calibration.

The position of the IMU on the handle and the IMU on the distal end are represented as:

^{g}Tᵢₘᵤ_{₁}=^{g}T₀×⁰T₁×¹T₂ (9)

^{g}Tᵢₘᵤ_{₂}=^{g}T⁰×⁰T₁×¹T₃×³T₄ (₁₀)

Wherein the readings from the IMUS 18 are with respect to gravity, g. Rᵢₘᵤ are determined by fusion of the LED pose and IMU readings. 1R₂ and ³R₄ are provided by calibration or set during assembly. The position and rotation of the array is then given by:

¹T₃=¹T₂×^{g}Tᵢₘᵤ_{₁}⁻¹×^{g}Tᵢₘᵤ_{₂}×³T₄⁻¹ (11)

¹R₃=¹R₂×^{g}Rᵢₘᵤ_{₁}⁻¹×^{g}Rᵢₘᵤ_{₂}×³R₄⁻¹ (12)

The result is a position or pose of the center of the ultrasound image, T₅:

⁰T₅=⁰T₁×¹T₃)×³T₅ (13)

³T₅ is based on calibration or set during assembly relative to ¹T₃, and ¹T₃ incorporates the pose tracking form the IMUs.

The processor 26 is configured to fuse pose information from multiple sources in one embodiment, One example is the fusion used above of IMU 18 and LED in the laparoscope. In another example, image processing from an x-ray image (see objective function of equations (3) or (4) above), image processing from a camera of LEDs (see objective function of equation (5) above), image processing from ultrasound, optical camera tracking, electromagnetic measures, and/or IMU outputs are combined. In one embodiment, the combination as provided in equation (6) is used. The poses from different sources are combined, such as averaged (e.g., average position along a given axis from multiple sources). In another embodiment, different parts of the pose are selected from different sources. For example, IMUs provide more accurate rotation and depth position information, and image processing from ultrasound and/or x-ray provide more accurate 2D position. The rotation and depth position pose from the IMU and the 2D position from the x-ray are used. The corresponding measures from each are combined to provide the pose.

In another embodiment, IMU measurements are used to assist in image processing to determine the pose. The IMU measurements limit the search space. For example, less accurate translation information from the IMU 18 is used to limit the position search in image processing of x-ray or ultrasound data. The search space for the pose is limited by the IMU output.

Other feedback systems may be used. The image processing may include pose information for a phase from the inertial measurement unit based on feedback from a previous pose. A position estimate is repetitively obtained by integration of these pose parameters over time based on a quasi-static motion model, which is defined by the IMU pose or pose measurements at the phase. An estimate based on IMU measures alone may include a large uncertainty due to accumulation of bias errors during the integration process. Feedback is used to remove or reduce the bias errors.

In an embodiment that uses the IMU 18, the search space of parameters of the transformation to be estimated from X-ray images may be reduced to T(xₜ)εSE³, where the transformation is only parameterized by the translational parameters, xₜε [symbol] ³. The rotation is parameterized by the more accurate IMU rotational measurements or outputs. FIG. 9 shows the processor 26 using the IMU 18 (e.g., MEMS-INS 34) to provide information to image processing 30 for pose recovery. The pose is recovered using fusion of information from X-ray image processing 32 of an x-ray image 30 and IMU 18 (MEMS-INS 34) sources. The instantaneous measurements of the IMU 18 (e.g., accelerometer, gyroscope, and/or magnetometer outputs) are input to the fusion 36 with the pose determined by the image processing 32. The fusion 36 combines information from the MEMS-INS 34 (e.g., rotation) and the x-ray image processing 32 (e.g., translation) to determine a pose 38. Each ultrasound frame acquired by the array is tagged with a correct pose 38 with respect to a reference coordinate system.

For the feedback, the information sharing between X-ray imaging and IMU sensors is bi-directional. The pose based on x-ray image processing and IMU outputs is feedback. The x-ray image processing uses IMU outputs. The pose uses IMU outputs and the x-ray image processing output. Bi-directional information sharing is used to refine estimates of the pose.

The pose 38, based in part on the image processing 32, is used to predict 40 a current phase of a physiological cycle. The fusion 36 outputs a low frequency, high certainty pose measurement 40 used as feedback to reset the drift errors associated with MEMS-INS sensors. The output is tied to the phase of the physiological cycle and is application dependent. For instance, in a cardiac application, the output may be given out at the phase of the cardiac cycle when the motion of the coronaries is the minimum. The pose is passed as a low frequency inertial ego-state (i.e., once per cycle anatomy pose or phase for the pose) to the MEMS-INS 34. The previous pose is used to estimate the state of a cardiac cycle. The derived linear acceleration and/or angular acceleration from the MEMS-INS 34 are triggered based on the phase. When a particular phase of the cycle is reached, the IMU (e.g., MEMS-INS) 34 is triggered to indicate the IMU-based pose at that phase. The values from previous repetitions of the phase are averaged or low pass filtered 42 to determine quasi-static components 44. The quasi-static components 44 are pose information from a model. The model is fit based on the outputs of the filter for that phase, but may be based on the pose at that phase based on the filtering.

The quasi-static components 44 are input as the ego-state of the anatomy of interest (e.g., heart) for the image processing 32. The state of other objects (e.g., anatomy or devices, the state of the environment or static objects (e.g., bone), and the x-ray image with the markers (25) are processed to determine the pose or at least some components (e.g., translation) of the pose. Any image processing may be used, such as described above.

In one embodiment, the final corrected pose estimate of the sensor fusion block, which is parameterized by 6 components, is constructed by picking each parameter from the measurement with the least uncertainly in that parameter. For instance, in a scenario with the ultrasound catheter 23 being imaged by X-ray, the two parameters for out-of-plane rotation are picked from the estimate provided by IMU, the in-plane translation and in-plane rotation parameters are picked from the X-ray pose estimate, and the out-of-plane translation is picked from the ultrasound decorrelation measurement. Under this scenario, each of these provide the least uncertainly in the respective parameter.

In another embodiment, the exchange of information between these sources of measurements provides a better estimate. The fusion 36 assumes a motion model (e.g., quasi-static) and formulates the pose estimation as a prediction-correction problem. The estimated measurements from each of the sources are used to iteratively correct the predicted positions to minimize the uncertainty in the corrected pose. The sensor fusion block may use an L2 norm (extended Kalman filter (EKF) approach) to determine the uncertainty in each of the parameters, for example, to determine a corrected pose within a desired level of uncertainty and a temporal resolution matching that of image acquisition. The iterative prediction-correction approach is not limited to this norm, and any other suitable norm, such as L₁, may be used as well. The trade-off between norms is between robustness to noise and computational cost of the problem.

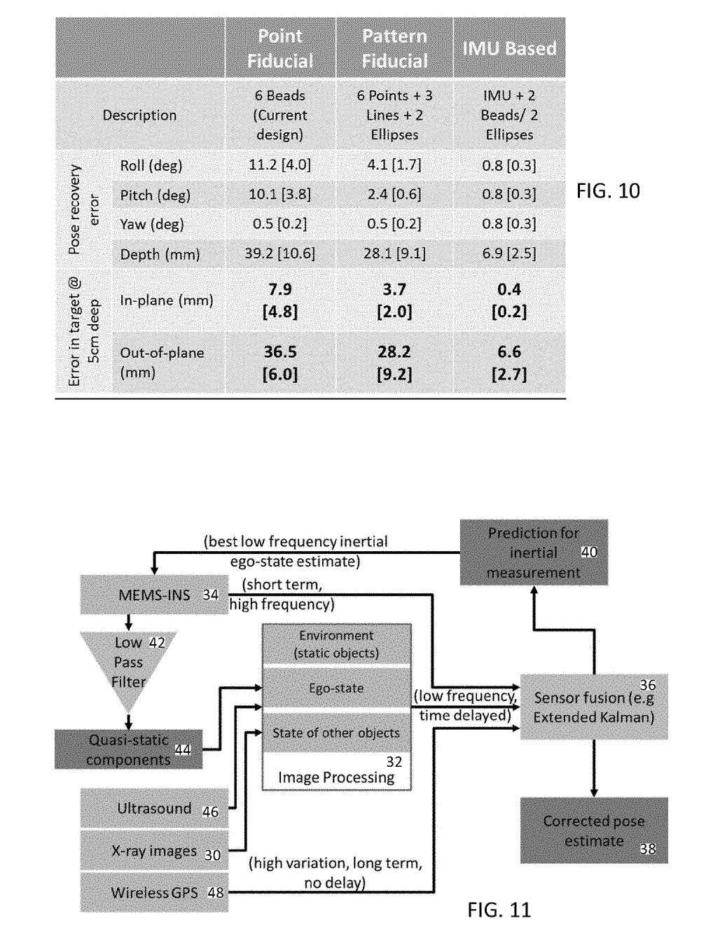

When a mono-plane X-ray image is used, the reduced search space due to input of the quasi-static component is of a high importance for accurate pose estimation because out-of-plane rotation and depth estimation from a single X-ray image is extremely difficult. In comparison, when only a subset of parameters are to be estimated (e.g. the depth), the robustness and accuracy may be significantly boosted by removing the complicating factors from other unknown parameters (e.g. rotations). For example, in the case of X-ray, some parameters such as the predicted rotations along with depth estimated from inertial measurement are applied. The x-ray images are then use to correct for errors in these predictions. Since inertial units are significantly accurate in estimating angulations, but not depth, the amount of corrections in angulation may be insignificant compared to that of depth. An example comparison on the achievable accuracy by using point fiducials, pattern fiducials, and pattern fiducials+IMU sensors is shown in FIG. 10. The accuracy values are obtained by simulating using a noise model with fiducial error of 0.27 mm. The 90th percentile of errors is shown with medians in brackets.

In case of a multi-detector setup or when the mono-plane system observes the catheter 23 from multiple viewpoints, a depth estimate from the back-projection of individual images may further constrain the search space of translational parameters, xₜ, based on the known uncertainty model of these depth estimates.

FIG. 11 shows pose recovery by the processor 26 with additional inputs for the image processing 32. A combination of information from multiple sensor and image modalities (x-ray 30, ultrasound 46, and indoor wireless 48) are utilized to accurately determine the pose of the ultrasound catheter 23. The catheter 23 is utilized in the presence of an X-ray imager. In the absence of such imager, the input of position and angulation (with some level of uncertainty) from pose recovery from X-ray is not available. In this case, the full pose recovery based on the fusion 36 may continue using the information from other sensors. This is also valid in case the X-ray source is turned on intermittently.

In the approaches of FIGS. 9 and 11, different pose information is available at different rates or times. MEMS-INS sensors 34 (IMU 18) based on a combination of accelerometers and gyroscopes provide high frequency measurements. These measurements may be decomposed into orientation estimates and position estimates. The orientation estimates are accurate and robust, but the position estimates are valid only for short term and have significant errors thereafter. The ultrasound image itself may provide pose estimates. Due to the process of image acquisition and image processing, these measurements are time delayed and have low update rates. Image processing may use a low pass filtered quasi-static components 44 of acceleration measurements for processing. This is to assign a directionality component to the distance measurement obtained using the correlation or matching to find the pose in the image processing 32. Measurements from wireless “GPS” do not have time delay and typically do not drift in the long-term, but have high uncertainty in the measurements. The measurements from the pose recovery from the X-ray source 30 by detection and tracking of radio-opaque markers (25) may have low update rates, but are of high accuracy with respect to in-plane parameters (i.e. in-plane translations and rotation). Further, the x-ray-based pose may be intermittent as in a typical clinical environment it is preferable to limit the X-ray exposure of both the clinician and patient. The measurements from these various sensors are input to the sensor fusion block. Any combination of inputs from IMU and the ultrasound images, with or without other inputs, may be used. Given the different frequencies, the Kalman filter, other expectation maximization, or other image process determines the pose at a higher frequency than the lowest frequency.

The image processor 32 may include an input from a camera, such as from capture of a pattern of LEDs. In other embodiments, the image of the LEDs captured by the camera are processed in a different way by the processor 26 to determine the pose. The pose may be determined with or without inputs from other position sensors (e.g., IMUs 18).

In one embodiment, post-process filtering is used. A nonlinear discrete time system with additive noise is represented as:

x(k+1)=F(x(k))+v(k) (14)

y(k)=H(x(k))+w(k) (15)

where x(k) is the state, y(k) is the measurement, and v(k) and w(k) as independent and identically distributed noises. The time system may be simplified to the form:

z(k)=η(z(k−1),y(k)) (16)

\hat{x}(k)=ζ(z(k),y(k)) (17)

The function η(•) and ζ(•) are parameterized by functions F(•), H(•) and statistics of x(k), v(k) and w(k). These take observations y(k) and produce an estimate \hat{x}(k), based on its internal state z(k) and the algorithms used. For Extended Kalman Filter, z(k) includes the estimated mean and covariance. For Partical Filters, z(k) includes all the particles that are used in the computation of the filter and their weights.

The estimate \hat{x}(k) is the typical representation of the 6 degrees of freedom of SE³ space. The internal state z(k) is composed of the position, velocity, and acceleration of the inertial frame described in SE³ space, which is dependent on parameters used to describe x(k) that is,

z(k)=[x(k),x{dot over (()}k),x{umlaut over (()}k)]^{T}

and state-space model is:

$$A = [ \begin{matrix}1 & Δ t & \frac{1}{2} Δ {t}^{2} \\ 0 & 1 & Δ t \\ 0 & 0 & 1\end{matrix} ] \qquad ( 18 )$$

The observations y(k) are determined by the particular embodiment of the system. In an embodiment that does not use an IMU, the observations are the output of optimizer (i.e., the rigid transformation T(x)).

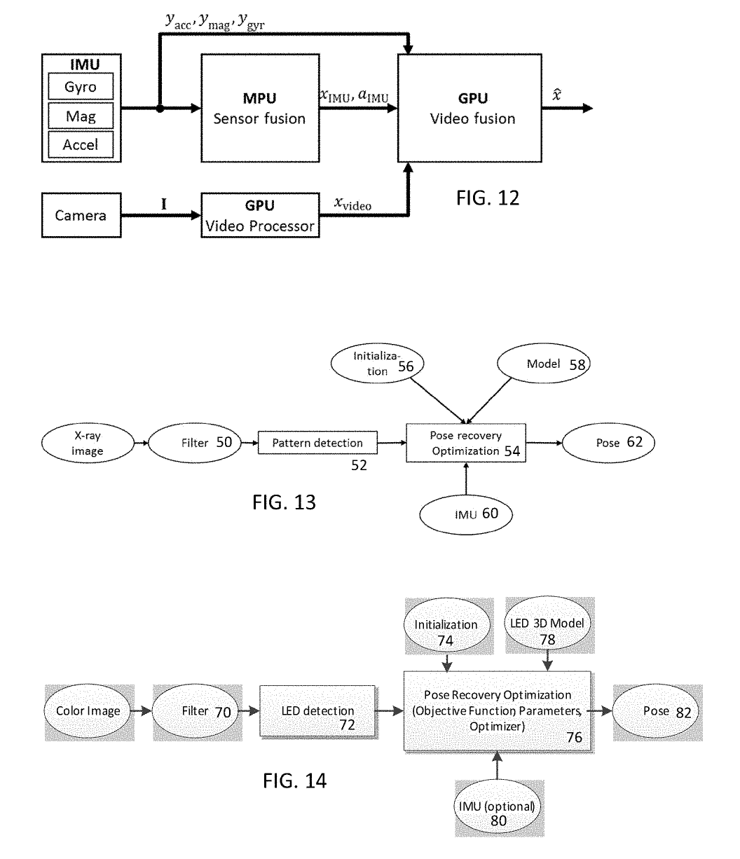

In an embodiment that uses an IMU 18, the observations y(k) are the output of optimizer (i.e., the rigid transformation T(x) and the accelerometer and gyroscope readings of the IMU). Since an IMU 18 rate may be an order of magnitude higher than the rate of video, a multi-stage multi-rate may be deployed as shown in FIG. 12, The observations arrive at different rates, and the observation model is changed accordingly. For time samples when the transformation information T(x) from video is available, the observation vector is defined as:

y(k)=[k_{video},y_{acc},y_{mag},y_{gyr},x_{IMU},α_{IMU}]^{T} (19)

For time samples when the transformation information T(x) from video is not available, the rows and columns corresponding to x_{video} in the observation vector and observation model matrix are set to 0.

For the above cases, the vectors of the observation vector are as follows: x_{video} is the parameterization of the transformation T(x) output from the video processor, y_{acc} is the acceleration readings from IMU 18, y_{mag} is the magnetometer from IMU, if available, y_{gyr} is the gyroscope readings readings from IMU, if available, x_{IMU} is the rotational component of the transformation output from the IMU feedback loop, and α_{IMU} is the linear acceleration output from the IMU feedback loop. In an embodiment that uses an IMU, the search space of parameters of the transformation may be reduced to T(xₜ)εSE³, where the transformation is only parameterized by the translational parameters, xₜε [symbol] ³. Additionally, information provided by the depth map (e.g., from structured light camera, time of flight camera or dense stereo reconstructions) may further constraint the search space of translational parameters, xₜ based on the known uncertainty model of these depth maps.

Referring again to FIG. 1, the display 28 is a monitor, LCD, projector, plasma display, CRT, printer, or other now known or later developed devise for outputting visual information. The display 28 receives images, graphics, or other information from the processor 26, memory 12, or ultrasound system 14. A display buffer outputting to the display 28 configures the display 28 to display an image.

One or more images representing a region of the patient are displayed. The image may represent the pose. For example, a location and orientation of the catheter 23 in an x-ray image are highlighted (e.g., colored or graphic overlay). With an adjacently displayed ultrasound image, the user may perceive the field of view relative to the x-ray image. As another example, the pose is used to transform coordinates between the ultrasound imaging system 14 and another imaging system (e.g., x-ray). The images from both modalities are combined or fused using the transformation.

The memory 12 is a graphics processing memory, video random access memory, random access memory, system memory, cache memory, hard drive, optical media, magnetic media, flash drive, buffer, database, combinations thereof, or other now known or later developed memory device for storing data or video information. The memory 12 is part of an imaging system (e.g., ultrasound system 14), part of a computer associated with the processor 26, part of a database, part of another system, or a standalone device.

Any type of data may be stored, such as medical image data (e.g., ultrasound and x-ray). The memory 12 stores datasets (e.g., frames) each representing a three-dimensional patient volume or a two-dimensional patient area. The patient volume or area is a region of the patient, such as a region within the chest, abdomen, leg, head, arm, or combinations thereof. The patient area or volume is a region scanned by the ultrasound system 14. Position data, such as camera images, IMU measurements, or other pose information is stored. The data represents the patient at one time or represents the patient over time, such as prior to or during treatment or other procedure.

Alternatively or additionally, the memory 12 or other memory is a non-transitory computer readable storage medium storing data representing instructions executable by the programmed processor 26 for pose recovery. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone, or in combination. Likewise, processing strategies may include multiprocessing, multitasking, parallel processing, and the like.

In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

FIGS. 13 and 14 show two example methods. FIGS. 9 and 11 show example methods as implemented by configuration of the processor. The methods of FIGS. 9 and 11 may be performed by other devices. Additional, different, or fewer acts than shown in FIGS. 9, 11, 13, and/or 14 may be used. The methods are implemented by the system of FIG. 1 or a different system.

FIG. 13 shows a flow chart of one embodiment of a method for pose recovery using an x-ray image and IMU. First, the X-ray image is filtered 50 to enhance the radio-opaque pattern to be detected 52. The detected pattern is input to optimization 54 of the pose recovery. Position estimation is initialized 56 with either the result from the previous frame, or parameters as detected in a multi-detector setup. Optimization 54 of the 3D pose minimizes the re-projection error that compares the filtered image with a 2D projection of the 3D CAD model 58 of the pattern. In addition, information from other sensors, such as an IMU system, is utilized 60 to provide orientation. The orientation constrains the search space of the position of the target pattern for an enhanced accuracy, especially in the depth direction. The recovered pose 62 based on the optimization is output,

In another embodiment, an ultrasound catheter 23 with a compact radio-opaque pattern and one or more inertial sensor is used to recover the pose (position and/or orientation) of the array with respect to a reference coordinate system. The radio-opaque pattern includes one or more fixed geometric shapes including lines and/or conics (e.g., circles, ellipses, etc.). The 3D pose of a target object is recovered by fusion of information from several sources including by not limited to X-ray images, the inertial sensors, ultrasound echo data (IQ or B-mode), and/or wireless narrowband signals.

For use with a multi-detector X-ray system, the image processing is duplicated. An increase in the coverage of the target object by multiple X-ray imagers decreases error in pose recovery and increases robustness to occlusion. A surface point cloud and/or analytical description of the radio-opaque pattern obtained from one or more X-ray images is used to initialize pose estimation. A pose may be recovered from only a partial view of the pattern of radio-opaque markers (25) where enough of the markers (25) are visible to allow solution.

A combination of readings from these multiple sensors is used to refine orientation in pose recovery and/or optimize search space parameters for Z-depth (distance along the view direction of the X-ray system) in translation. The pose recovered using the method is used to track the location of the ultrasound image with respect to an arbitrary reference coordinate system. The pose may be used to reconstruct a 3D volume of ultrasound from a freehand sweep of target anatomy. The pose indicates the relative position of the acquired frames of ultrasound data. The recovered pose may be used in coverage analysis. A boundary or model of the patient and the pose are used to indicate to the user the fraction of the target anatomy that has been scanned or remains to be scanned to ensure sufficient image acquisitions have been made.

FIG. 14 shows a flow chart of one embodiment of a method for pose recovery using camera capture of an LED pattern. First, the 2D color camera image is filtered 70 to emphasize the LED pattern relative to another signal. The LED patter is detected 72 by image processing. Position estimation may be initialized 74 with either the result from the previous frame, or parameters as detected in an optional depth camera. Optimization 76 of the 3D pose minimizes the re-projection error that compares the filtered image with a 2D projection of the 3D CAD model 78 of the LED pattern. Optionally, other sensors, such as an IMU system 80, may be integrated to provide orientation and/or constrain the search space of the position of the target pattern. The optimized or recovered pose 82 is output.

In another embodiment, the pose is recovered with respect to a reference coordinate system using a monocular vision system and a compact illuminated pattern. The illuminated pattern has one or more fixed geometric shapes including lines and/or conics (e.g., circles, ellipses, etc.). The 3D pose of a target object is recovered by minimizing the difference between a frame of the illuminated pattern, captured by the camera, and the 2D projection of the known geometric model of the pattern. The coverage of the target object may be increased by using multiple cameras, decreasing error in pose recovery and increasing robustness to occlusion. A surface point cloud of the illuminated pattern obtained from a depth camera is used to initialize pose estimation. A combination of readings from one or more IMU sensors may be used to refine orientation in pose recovery and/or optimize search space parameters for Z-depth (distance along the view direction of the vision system) in translation.

The pose recovered using the method is used to track the location of the ultrasound image with respect to an arbitrary reference coordinate system. The pose may be used to reconstruct a 3D volume of ultrasound from a freehand sweep of target anatomy. The recovered pose may be used in coverage analysis to indicate to the user the fraction of the target anatomy that has been scanned or remains to be scanned to ensure sufficient image acquisitions have been made. In some embodiments, the illuminated pattern is attached to a straight needle-like interventional device (e.g., biopsy needle, ablation device, etc.) to provide a pose estimate of the tip of the device with respect to a fixed coordinate system. The attachment may be accomplished by a clip-on part that affixes to the shaft of the needle-like device or incorporated as integral part of the device such as the handle. IMUs may alternatively or additionally be attached in a fixed known configuration relative to the illuminated pattern as well as to the shaft of the needle-like device for pose recovery. The pose may be recovered from a partial view of the 3D illuminated pattern.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A system for pose recovery of an ultrasound transducer, the system comprising:
an inertial measurement unit connected with the ultrasound transducer;
a position sensor positioned to sense the ultrasound transducer; and
a processor configured to determine the pose of the ultrasound transducer based on outputs from the position sensor and the inertial measurement unit.

2. The system of claim 1 wherein the inertial measurement unit comprises an accelerometer, a gyroscope, a magnetometer, or a combination thereof.

3. The system of claim 1 where the ultrasound transducer is in a catheter, and wherein the inertial measurement unit is in the catheter within a centimeter of an array of the ultrasound transducer.

4. The system of claim 3 wherein the ultrasound transducer includes a plurality of radio-opaque markers, and wherein the position sensor comprises an x-ray imager configured to image the radio-opaque markers.

5. The system of claim 1 wherein the ultrasound transducer comprises a handheld transducer with a line, curve, or area shaped pattern of light emitting diodes, and wherein the position sensor comprises a camera with a filter configured to isolate information from a range of frequencies of the light emitting diodes.

6. The system of claim 1 wherein the ultrasound transducer comprises a laparoscope, and wherein the inertial measurement unit is positioned in a portion of the laparoscope with an array, the portion being rotatable relative to another part of the laparoscope.

7. The system of claim 1 wherein the processor is configured to determine the pose with the output from the inertial measurement unit at a higher frequency than the output from the position sensor, the pose determined at the higher frequency.

8. The system of claim 4 wherein the processor is configured to determine the pose from image processing of an image from the x-ray imager, the image processing including pose information for a phase from the inertial measurement unit based on feedback from a previous pose.

9. The system of claim 1 wherein the position sensor comprises an ultrasound scanner connected with the ultrasound transducer or a wireless positioning system.

10. The system of claim 4 wherein the processor is configured to determine the pose with the output of the inertial measurement unit limiting a search space for the pose.

11. The system of claim 1 wherein the processor is configured to initialize the determination of the pose with a surface point cloud.

12. The system of claim 1 wherein the processor is configured to determine the pose with an expectation maximization filter based on the outputs.

13. A system for pose recovery of an ultrasound transducer, the system comprising:
a handheld housing of the ultrasound transducer;
light emitting diodes positioned on the handheld housing, the light emitting diodes forming a geometric pattern of adjacent, visually connected light sources;
a camera positioned to capture an image of the light emitting diodes;
a filter configured to reduce signal in the image at frequencies different than emission frequencies of the light emitting diodes; and
a processor configured to determine the pose based on minimization of difference between the image output by the filter and two-dimensional projection from a model of the geometric pattern.

14. The system of claim 13 wherein the light emitting diodes are embedded in the handheld housing with the geometric pattern being distributed on a non-planar three-dimensional surface.

15. The system of claim 13 wherein the geometric pattern comprises one or more lines, curves, two-dimensional shapes, or combinations thereof of the visually connected light sources.

16. The system of claim 13 wherein the processor is configured to determine the pose with initialization from a previous estimate of the pose or a three-dimensional point cloud of at least part of the handheld housing.

17. The system of claim 13 further comprising an inertial measurement unit on the handheld housing, wherein the processor is configured to determine the pose based on the minimization and an output of the inertial measurement unit.

18. The system of claim 17 wherein the processor is configured to determine the pose based on the minimization with the output limiting a search of the minimization, refining the minimization with orientation as the output, or combinations thereof.

19. The system of claim 17 wherein the handheld housing comprises a laparoscope with the inertial measurement unit on a first part insertable within a patient and the light emitting diodes on a second part handheld while the first part is inserted in the patient.

20. A system for pose recovery of an ultrasound transducer, the system comprising:
a catheter having an array of acoustic transducer elements, an inertial sensor, and a radio-opaque marker;
an x-ray imager configured to image the catheter while in a patient;
a processor configured to determine a pose of the array with the image from the x-ray imager and an output of the inertial sensor.
